(19) United States
(12) Patent Application Publication
(10) Pub. No.: US 2022/0088074 A1
(43) Pub. Date: Mar. 24, 2022

(54) GENETICALLY MODIFIED GAMMA DELTA T CELLS AND METHODS OF MAKING AND USING

(71) Applicant: Regents of the University of Minnesota, Minneapolis, MN (US)

(72) Inventors: Beau Webber, Minneapolis, MN (US); Branden Moriarity, Minneapolis, MN (US); Emily Joy Pomeroy, Minneapolis, MN (US); Patricia Nicole Claudio Vazquez, Minneapolis, MN (US)

(21) Appl. No.: 17/429,452

(22) PCT Filed: Feb. 21, 2020
(86) PCT No.: PCT/US2020/019267

§ 371 Date: Aug. 9, 2021

Related U.S. Application Data
(60) Provisional application No. 62/808,504, filed on Feb. 21, 2019.

(51) Int. Cl. A61K 35/17; C12N 5/0783; A61P 35/00

(52) U.S. Cl. CPC A61K 35/17; A61P 35/00; C12N 5/0636

(57) ABSTRACT

Provided herein are genome-edited γδ T cells that exhibit an increased capacity to kill cancer cells, methods of producing genome-edited γδ T cells, and methods of treating or preventing a condition by administering genome-edited γδ T cells to a subject in need thereof.

### CROSS-REFERENCE TO RELATED APPLICATIONS

This application claims priority to U.S. Provisional Application No. 62/808,504, filed Feb. 21, 2019, which is incorporated herein by reference as if set forth in its entirety.

### STATEMENT REGARDING FEDERALLY FUNDED RESEARCH OR DEVELOPMENT

Not applicable.

### SEQUENCE LISTING

A Sequence Listing accompanies this application and is submitted as an ASCII text file of the sequence listing named “920171.00336 ST25.txt” which is 3 KB in size and was created on Feb. 19, 2020. The sequence listing is electronically submitted via EFS-Web with the application and is incorporated herein by reference in its entirety.

### BACKGROUND

In the United States alone 1.7 M people are predicted to be diagnosed with cancer in 2018, and of those, 609,000 will die (CDC, ACS). When you add in the burden of cancer treatment cost alongside the 35% mortality, it becomes clear that the advent of better, more affordable therapies is urgently needed. One therapy that has shown promising results for multiple cancer treatments is Adoptive T Cell Transfer (ATCT), wherein patient T cells are removed, manipulated, and expanded ex vivo, and re-infused into patients to target tumor sites. Several recent studies have shown that ATCT therapies have anti-tumor activity and are safe to use in human patients, supporting ATCT as a possible avenue for developing novel, efficacious cancer treatments (Ahmed, N., et al., 2009; Li, Z., et al., 2011). However, there are still pressing problems to be solved with ATCT therapies in the context of targeting both solid and blood cancers including improving their ability to target solid tumors, increasing overall efficacy, and preventing on-target, off-tumor immunoreactivity. Accordingly, there remains a need in the art for improved therapeutic compositions and methods for targeting and killing solid tumors and blood cancers.

### SUMMARY

This disclosure describes genome-edited γδ T cells, methods of making genome-edited γδ T cells, and methods of using the genome-edited γδ T cells.

In a first aspect, provided herein is a method for editing a genome of an activated γδ T cells. The method can comprise or consist essentially of (a) providing a cell sample comprising T cells, T cell subsets and/or T cell progenitors; (b) separating γδ T cells or a γδ T cell subset to thereby provide enriched γδ T cells; (c) activating enriched γδ T cells using one or more modulatory agents to thereby provide activated γδ T cells; (d) genetically modifying the activated γδ T cells to thereby provide genetically modified T cells comprising one or more modifications in at least one gene selected from IL-17A (Interleukin 17A), DGKA (Diacylglycerol Kinase Alpha), DGKZ (Diacylglycerol Kinase Zeta), PD1 (programmed cell death 1), TRGC1 (T-cell Receptor Gamma Constant-I), TRGC2 (T-cell Receptor Gamma Constant-2), TRDC (T-cell Receptor Delta Constant), PD-L1 (Programmed death-ligand 1; also known as CD274), and CISH (Cytokine-inducible SH2-containing protein), or any combination thereof. The method can further comprise expanding the genetically modified γδ T cells to thereby provide an expanded population of genetically modified γδ T cells. The one or more modulating agents can be selected from CD28, CD3, and Concanavalin A. The genetically modified γδ T cells can further comprise a chimeric antigen receptor comprising an extracellular domain capable of binding to an antigen, a transmembrane domain, and at least one intracellular domain. The antigen can be a tumor antigen. The extracellular domain capable of binding to an antigen can be a single chain variable fragment of an antibody that binds to the antigen. The step of genetically modifying can comprise introducing a nuclease or a nucleic acid encoding a nuclease into the γδ T cell. The nuclease can comprise Cas9. The step of genetically modifying can comprise introducing a chemically modified guide RNA (gRNA) into the γδ T cell. The chemically modified gRNA can comprise 2′-O-methyl (M), 2′-O-methyl-3′-phosphorothioate (MS), or 2′-O-methyl-3′-thiophosphonoacetate (MSP).

In another aspect, provided herein is a genome-edited γδ T cell that comprises one or more mutations in a gene selected from IL-17A (Interleukin 17A), DGKA (Diacylglycerol Kinase Alpha), DGKZ (Diacylglycerol Kinase Zeta), PD1 (programmed cell death 1), TRGC1 (T-cell Receptor Gamma Constant-1), TRGC2 (T-cell Receptor Gamma Constant-2), TRDC (T-cell Receptor Delta Constant), PD-L1 (Programmed death-ligand 1), and CISH (Cytokine-inducible SH2-containing protein), or any combination thereof. In some cases, the genome-edited γδ T cell further comprises a chimeric antigen receptor comprising an extracellular domain capable of binding to an antigen, a transmembrane domain, and at least one intracellular domain. The antigen can be a tumor antigen. The extracellular domain capable of binding to an antigen can be a single chain variable fragment of an antibody that binds to the antigen. In some cases, the gene is deleted. In some cases, the gene comprises a point mutation. In some cases, the genome-edited γδ T cell further comprises an exogenous gene. The genome-edited γδ T cell can exhibit increased capacity to kill cancer cells relative to a non-genome-edited γδ T cell.

In another aspect, provided herein is a method for treating or preventing a disease in a subject. The method can comprise or consist essentially of administering to the subject a composition comprising the genome-edited γδ T cell as provided herein. The disease can comprise cancer or a precancerous condition.

### BRIEF DESCRIPTION OF THE DRAWINGS

FIGS. 1A-1B are schematics illustrating a general chimeric antigen receptor (CAR) structure (A) and a CAR-expressing γδ T cell, in which the CAR's antigen binding region comprises an scFv of an antibody that binds to the target antigen.

FIG. 2 is a schematic illustrating isolation of γδ T cells using variable methods.

FIGS. 3A-3B present a schematic illustration GDTC isolation and expansion (A) and purity of GDTCs isolated from PBMCs and post-CD3 stimulation.

FIG. 4 demonstrates electroporation efficiency of γδ T cells.

FIGS. 5A-5E present gene targeting data.

FIG. 6 lists genomic targets and exemplary guide sequences (SEQ ID NOs:1-9).

FIGS. 7A-7B demonstrate the composition of Vδ1⁺ and Vδ2⁺ subsets within in vitro isolated and expanded γδ T cells. (A) Frequency of Vδ1⁺ and Vδ2⁺ γδ T cells in CD4/CD8 depleted PBMC before stimulation. (B) Frequency of Vδ1⁺ and Vδ2⁺γδ T cells following stimulation and expansion with either ConA, CD3/CD28 Dynabeads, or Zolendronate.

FIG. 8 demonstrates optimizing CRISPR/Cas9 gene knockout in human γδ T cells. (A) Purified γδ T cells were stimulated with either anti-CD3 antibody alone (OKT3) or anti-CD3 and anti-CD28 antibodies (CD3/CD28). Electroporation of CRISPR sgRNA+Cas9 mRNA (1.5 Cas9) was performed at either 48 hours or 72 hours (hrs) post-stimulation as indicated, with pulse alone or GFP serving as no edit controls.

FIG. 9 demonstrates knockout of immunosuppressive molecules in human γδ T cells. Purified γδ T cells were stimulated with anti-CD3 and anti-CD28 antibodies (CD3/CD28). Electroporation of Cas9 mRNA and CRISPR sgRNAs targeting the genes encoding Programmed Death-ligand 1 (PD-L1) (SEQ ID NO:6) and Interleukin-17 (SEQ ID NO:7) were performed at 72 hr post-stimulation. Editing at the genomic target was assessed after expansion by Sanger sequencing and TIDE analysis.

FIGS. 10A-10E demonstrate CRISPR/Cas9 editing of PD1, CISH, and TRDC in human γδ T cells. (A) Frequency of targeted indel creation at PD1, CISH, and TRDC in γδ T cells as measured by Sanger sequencing and TIDE analysis. (B) Quantification of protein loss for PD1, CISH, and TRDC in edited γδ T cells. (C) Representative flow cytometry expression of VM and Vδ2 (together, total TRDC expression) in pulse control and CRISPR/Cas9 edited γδ T cells. (D) Representative flow cytometry expression of PD1 staining in pulse control and CRISPR/Cas9 edited γδ T cells. (D) Representative flow cytometry expression of TRDC expression in pulse control and CRISPR/Cas9 edited γδ T cells. (E) Western blot analysis of CISH KO in pulse control (WT) and CRISPR/Cas9 edited γδ T cells.

FIG. 11 demonstrates targeted integration of a chimeric antigen receptor (CAR) at the AAVS1 safe harbor locus using CRISPR/Cas9 and rAAV-mediated donor delivery. Human γδ T cells were activated using either CD3/CD28 dynabeads or zolendronate and electroporated with Cas9 mRNA and a sgRNA targeting AAVS1. Following electroporation, γδ T cells were transduced with rAAV encoding a gen3 (third generation) or gen4 (fourth generation) Mesothelin-reactive CAR flanked by AAVS1 homology arms. Following expansion, expression of the CAR construct was measured by flow cytometry for linked RQR8 protein.

FIG. 12 demonstrates cytotoxicity of CRISPR/Cas9 engineered human γδ T cells. Human γδ T cells were activated with CD3/CD28 dynabeads, followed by lentiviral transduction with a gen3 chimeric antigen receptor reactive to mesothelin. Control un-transduced (UT) and CAR transduced γδ T cells were electroporated with Cas9 mRNA and sgRNA targeting either PD1 or CISH alone, or PD1 and CISH combined. Pulse only cells received no sgRNA. Following expansion, engineered γδ T cells were co-incubated with the mesothelin expressing ovarian cancer line A1847 at the indicated effector to target (E:T) ratios. Cytotoxicity was measured by loss of A1847 luciferase luminescence 24 hours following co-culture as normalized to A1847 that were not incubated with γδ T cells.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the appended claims.

### DETAILED DESCRIPTION

All publications, including but not limited to patents and patent applications, cited in this specification are herein incorporated by reference as though set forth in their entirety in the present application.

This disclosure describes genome-edited γδ T cells (also known as gamma delta (γδ) T lymphocytes), methods of making such cells, and methods of administering such cells. γδ T cells are capable of infiltrating solid tumors and directly killing transformed cells in a largely MHC-independent fashion via recognition of stress-induced antigens and metabolites. Since γδ T cells are the fraction of tumor infiltrating lymphocytes most highly correlated with positive outcomes from anti-cancer immunotherapies, γδ T cells may be better than αβ T cells (alpha-beta T cells) for infiltrating solid tumor microenvironments and efficient tumor-cell killing. Accordingly, γδ T cells provide an ideal platform for the development of immunotherapies against both blood and solid tumors. The genome edited cells and methods provided herein are based at least in part on the inventors' development of locus-specific CRISPR-Cas-mediated integration of chimeric antigen receptors (CARs) into γδ T cells to promote the inherent anti-tumor activity of γδ T cells. As compared to randomly integrating virus-based gene delivery, locus-specific CRISPR-Cas-mediated integration provides for improved expression levels of the CAR as well as reduced risk of undesirable side effects from random-integration events. By targeting the endogenous gamma-delta T cell receptor, we can achieve endogenous levels of receptor expression to decrease on-target/off-tumor reactivity while simultaneously preventing any random-integration.

Accordingly, in a first aspect, provided herein is a genetically modified gamma delta T lymphocyte (gamma delta T cell). Preferably, the genome-editing gamma delta T cell includes a modification of one or more genes selected from IL-17A (Interleukin 17A), DGKA (Diacylglycerol Kinase Alpha), DGKZ (Diacylglycerol Kinase Zeta), PD1 (programmed cell death 1), PD-L1 (Programmed death-ligand 1 also known as CD274), and CISH (Cytokine-inducible SH2-containing protein), or any combination thereof. As used herein, the term “γδ T cell” (gamma delta T cell) refers to T lymphocytes that express a gamma delta T cell receptor such as, for example, Vy9Vδ2 (gamma 9 delta 2) T cell receptor. γδ T cells represent a small subset of T lymphocytes within peripheral blood in humans. γδ T cells are characterized by production of abundant pro inflammatory cytokines such as IFN-gamma, potent cytotoxic effective function, and MHC-independent recognition of antigens. γδ T cell marker characteristics include, without limitation, CD3, CD4, CD8, CD69, CD56, CD27 CD45RA, CD45, TCR-Vg9, TCR-Vd2, TCR-Vd1, TCR-Vd3, TCR-pan g/d, NKG2D, monoclonal chemokine receptor antibodies CCR5, CCR7, CXCR3 or CXCR5 or combinations thereof.

In some embodiments, the genome-edited γδ T cell includes a modification in a coding region of the genome (for example, a gene) or a noncoding region of the genome. In some embodiments, a portion of genomic information and/or a gene may be deleted. In some embodiments, a portion of genomic information and/or a gene may be added. In some embodiments, the genomic information and/or the gene that is added is exogenous. In some embodiments, “exogenous” genomic information or an “exogenous” gene may be genomic information or a gene from a non-gamma delta T cell. In some embodiments, “exogenous” genomic information or an “exogenous” gene may be artificially generated including, for example, nucleic acids encoding a chimeric antigen receptor (CAR) or a marker gene. In some embodiments, a portion of genomic information and/or a gene may be altered, for example, by a mutation. A mutation may include, for example, a point mutation, a frameshift mutation, etc.

The genetic modification can alter expression or activity of the genome-edited γδ T cell. In some embodiments, the genome-edited γδ T cell may exhibit increased capacity to kill cancer cells relative to a non-genome-edited γδ T cell. In some embodiments, the genome-edited γδ T cell includes a modification that alters expression or activity of an inhibitory receptor relative to a non-genome-edited γδ T cell. For example, the genome-edited γδ T cell may comprise a mutation in one or more genes encoding an inhibitory receptor, whereby expression of the inhibitory receptor is decreased, partially or fully. The one or more genes encoding an inhibitory receptor can be selected from IL-17A (Interleukin 17A), DGKA (Diacylglycerol Kinase Alpha), DGKZ (Diacylglycerol Kinase Zeta), PD1 (programmed cell death 1), TRGC1 (T-cell Receptor Gamma Constant-1), TRGC2 (T-cell Receptor Gamma Constant-2), TRDC (T-cell Receptor Delta Constant), PD-L1 (Programmed death-ligand 1; also known as CD274), and CISH (Cytokine-inducible SH2-containing protein), or any combination thereof. Other inhibitory receptor genes include, without limitation, CD94-NKG2A, NKG2A, TIGIT, a member of the KIR2DL family (for example, KIR2DL1; KIR2DL2; KIR2DL3; KIR2DL4; or KIRDL5), a member of the KIR3DL family (KIR3DL1; KIR3DL2; or KIR3DL3), KLRG1, LILR, 2B4 (CD48), CD96 (Tactile), LAIR1, KLB1 (CD161), CEACAM-1, SIGLEC3, SIGLEC7, SIGLEC9, and/or CTLA4.

In some cases, the genetically modified γδ T cell is further modified to express a chimeric antigen receptor. As used herein, the term “chimeric antigen receptor (CAR)” (also known in the art as chimeric receptors and chimeric immune receptors) refers to an artificially constructed hybrid protein or polypeptide comprising an extracellular antigen binding domain of an antibody (e.g., single chain variable fragment (scFv)) operably linked to a transmembrane domain and at least one intracellular domain. Generally, the antigen binding domain of a CAR has specificity for a particular antigen expressed on the surface of a target cell of interest. For example, a T cell can be engineered to express a CAR specific for molecule expressed on the surface of a particular cell (e.g., a tumor cell, B-cell lymphoma). The antigen recognition region of the extracellular domain permits binding of the CAR to a particular antigen of interest, for example, an antigen present on a cell surface, and thereby imparts specificity to a cell expressing a CAR.

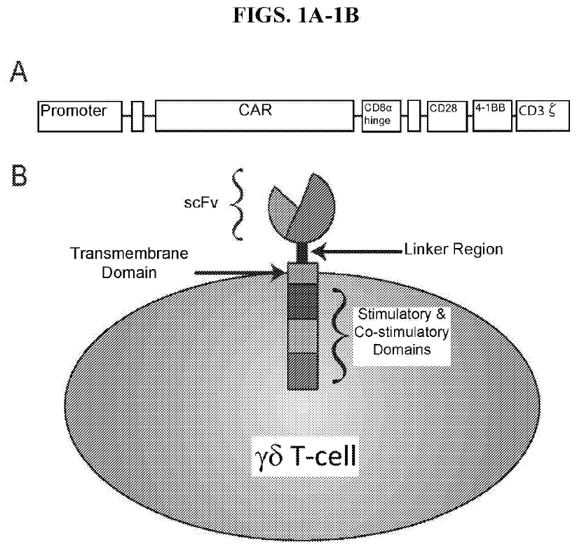

Referring to FIG. 1A, the CAR may comprise an extracellular domain (ectodomain) that includes an antigen recognition region, a transmembrane domain linked to the extracellular domain, and an intracellular domain (endodomain) linked to the transmembrane domain. The transmembrane domain can include a transmembrane region of a γδ T cell.

In some cases, extracellular domains are derived from antibodies (H chain and L chain) and variable regions of a TCR (TCRα, TCRβ, TCRγ, TCR δ), CD8α, CD8β, CD11A, CD11B, CD11C, CD18, CD29, CD49A, CD49B, CD49D, CD49E, CD49F, CD61, CD41, and CD51. The entire protein may be used effectively, and however, in particular, a domain capable of binding to an antigen or a ligand, for example, an extracellular domain of an antibody Fab fragment, an antibody variable region [V region of H chain (VH) and V region of L chain (VL)] or a receptor can be used. In certain embodiments, the extracellular domain comprises a single chain variable fragment of an antibody as illustrated in FIG. 1B. Examples of the antigen include, without limitation, a viral antigen, a bacterial antigen (particularly an antigen derived from an infectious bacterium), a parasite antigen, a cell surface marker on a target cell related to a certain condition (e.g. a tumor antigen), and a surface molecule of an immunity-related cell.

In some cases, the extracellular domain further comprises one or more of a signal peptide or leader sequence, and spacer. In addition, the present invention includes both a CAR comprising one extracellular domain and a CAR comprising two or more extracellular domains.

The intracellular domain is a molecule that can transmit a signal into a cell when the extracellular domain present within the same molecule binds to (interacts with) an antigen. Examples of intracellular domains include, without limitation, cytoplasmic sequences derived from a TCR complex and a costimulatory molecule, and any variant having the same function as those sequences. In some cases, the intracellular domain comprises a signaling peptide capable of activating a γδ T cell. The intracellular domain can further include a signaling domain of a γδ T cell membrane-bound signaling adaptor protein. Examples include but are not limited to: PI3K, ITK, Grb2, TRAF2, TRAF5, Siva, Jak1, Jak3, DAP10, CD3c, DAP12, PKC, LFA-1, Fyn, SHP-1, and SHP-2 (Ribeiro, S., et al., 2015).

The transmembrane domain may be derived from a natural polypeptide, or may be artificially designed. The transmembrane domain derived from a natural polypeptide can be obtained from any membrane-binding or transmembrane protein. For example, a transmembrane domain of a T cell receptor α or β chain, a CD3t chain, CD28, CD3c, CD45, CD4, CD5, CD8, CD9, CD16, CD22, CD33, CD37, CD64, CD80, CD86, CD134, CD137, ICOS, or CD154 can be used.

In some cases, a spacer domain can be arranged between the extracellular domain and the transmembrane domain, or between the intracellular domain and the transmembrane domain. The spacer domain means any oligopeptide or polypeptide that serves to link the transmembrane domain with the extracellular domain and/or the transmembrane domain with the intracellular domain. The spacer domain comprises up to 300 amino acids, preferably 10 to 100 amino acids, and most preferably 25 to 50 amino acids.

Methods of Making Genetically Modified GDTCs

In another aspect, provided herein is a method of producing of genetically modified gamma-delta Car T-cells. In some cases, the method comprises isolating gamma-delta T-cells, stimulating the isolated γδ T cells, expanding the stimulated γδ T cells, and introducing nucleic acids into the expanded, stimulated γδ T cells for genetic modification of the cells. In some cases, post-isolation stimulation of the γδ T cells comprises contacting the isolated cells with an antibody (e.g., OKT3 antibody) in a cell culture medium that contains IL-2 and IL-4 to facilitate expansion. Following the introduction of genetic modifications in the stimulated, expanded γδ T cells, the genetically modified γδ T cells are re-stimulated as described herein to specifically expand the genetically modified γδ T cells population. In some cases, the genetic modification comprises one or more modifications to a gene such as IL-17A, DGKA, DGKZ, PD1, PDL-1, CISH, or any combination thereof. The genetic modification can reduce or eliminate expression of targeted gene(s). In some cases, the gamma delta T cells comprises one or more modifications to a gene such as IL-17A, DGKA, DGKZ, PD1, PDL-1, CISH, or any combination thereof, and are further modified to express a chimeric antigen receptor.

γδ T cells can be isolated according to any appropriate method. For instance, wild-type gamma delta T cells can be isolated from peripheral blood mononuclear cells (PBMCs). PBMCs can be obtained from peripheral blood using any appropriate technique such as, for example, an ACK-lysis buffer protocol. For example, γδ T cells can be isolated using a commercially available kit such as the EasySep Human Gamma/Delta T Cell Isolation Kit from StemCell Technologies. In other cases, γδ T cells can be isolated by plating PBMCs in a culture medium containing Concanavalin A, IL-2, and IL-4 for about 1 week. Cells are further cultured in a cultured medium that does not contain Concanavalin A for an additional 7 days. Another isolation method comprises plating PBMCs in a culture medium containing Zolendronic Acid and IL-2 for about 2 days. The cells can be further cultured in a medium that does not contain Zolendronic Acid for an additional 12 days. In some cases, percent purity of the isolated γδ T cell population is determined using flow cytometry, Magnetic cell sorting, or another cell sorting method.

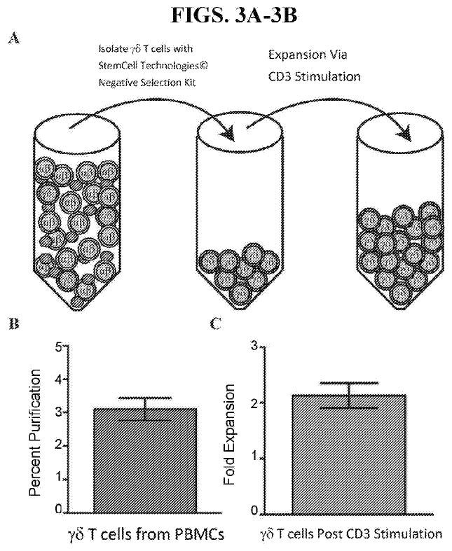

Following isolation, γδ T cells may be stimulated according to any appropriate protocol. In some cases, isolated γδ T cells are stimulated using Concanavalin A (Con A), a mannose/glucose-binding lectin isolated from Jack beans (Canavalia ensiformis) that acts as a T cell mitogen to activate the immune system, recruit lymphocytes, and elicit cytokine production. In other cases, isolated γδ T cells are stimulated with CD3, or CD3/CD28 antagonists which promotes rapid replication and expansion of the cells. Referring to FIG. 3B, isolated γδ T cells reach logarithmic growth about 3 days to about 5 days after stimulation with CD3 or CD3/CD28 antagonists. Alternatively, γδ T cells can be activated through direct stimulation with an antagonist to the γδ T cell receptor (TCR).

A γδ T cell is “genome edited” or “genetically modified” if the γδ T cell includes a modification to its genome compared to a non-genome edited γδ T cell. In some cases, a non-genome edited γδ T cell is a wild-type γδ T cell. As used herein, the terms “genetically modified” and “genetically engineered” are used interchangeably and refer to a prokaryotic or eukaryotic cell that includes an exogenous polynucleotide, regardless of the method used for insertion. In some cases, a γδ T cell has been modified to comprise a non-naturally occurring nucleic acid molecule that has been created or modified by the hand of man (e.g., using recombinant DNA technology) or is derived from such a molecule (e.g., by transcription, translation, etc.). A γδ T cell that contains an exogenous, recombinant, synthetic, and/or otherwise modified polynucleotide is considered to be an engineered or “genome edited” cell. Genetically editing or modifying a cell refers to modifying cellular nucleic acid within a cell, including genetic modifications to endogenous and/or exogenous nucleic acids within the cell. Genetic modifications can comprise deletions, insertions, integrations of exogenous DNA, gene correction and/or gene mutation.

The term “substantially pure cell composition of genetically modified gamma delta T cells and/or gamma delta T cell subsets” as used herein refers to a cell composition comprising at least 70%, more preferentially at least 90%, most preferentially at least 95% of genetically modified gamma delta T cells or gamma delta T cell subsets in the cell composition obtained by methods of the this disclosure.

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP, TALE, CRISPR/Cas, or base editor system as described herein. Thus, gene inactivation may be partial or complete.

To modify cells to comprise a CAR, a nucleic acid encoding a chimeric antigen receptor is introduced into a γδ T cell. Preferably, the CAR comprises an extracellular domain capable of binding to an antigen, a transmembrane domain, and at least one intracellular domain.

Various gene editing technologies are known to those skilled in the art. Generally, gene editing systems employ editing polypeptides, which are proteins that function to edit a nucleobase, nucleotide, or nucleoside, typically using single-stranded or double-stranded DNA breaks. As used herein, the term “edit” refers to the insertion or deletion of basepairs (called “indels”) and the conversion of one nucleobase to another (e.g., A to G, A to C, A to T, C to T, C to G, C to A, G to A, G to C, G to T, T to A, T to C, T to G). Gene editors include, without limitation, homing endonucleases, zinc-finger nucleases (ZFNs), transcription activator-like effector (TALE) nucleases (TALENs), clustered regularly interspaced short palindromic repeats (CRISPR)-associated proteins (e.g., Cas9), and nucleobase editors of base editor systems. Homing endonucleases generally cleave their DNA substrates as dimers, and do not have distinct binding and cleavage domains. ZFNs recognize target sites that consist of two zinc-finger binding sites that flank a 5- to 7-base pair (bp) spacer sequence recognized by the FokI cleavage domain. TALENs recognize target sites that consist of two TALE DNA-binding sites that flank a 12- to 20-bp spacer sequence recognized by the FokI cleavage domain. In some cases, gene editing comprises CRISPR-targeted, TALEN-targeted, or ZFN-targeted silencing of genes via methylation. Such gene editing techniques employ targeted DNA methylation to silence specific genes without altering the host genomic sequence. See, e.g., Lei et al., Nature Communications volume 8, Article number: 16026 (2017).

In some cases, gene editing is performed using an RNA-guided nuclease such as a CRISPR-Cas system, such as a CRISPR-Cas9 system specific for the target gene (e.g., an immunosuppressive gene, a co-stimulatory molecule) that is disrupted. For CRISPR/Cas-based gene editing systems, the nucleobase editors are generally Cas polypeptides and variants thereof. Cas9 is a nuclease that targets to DNA sequences complementary to the targeting sequence within the single guide RNA (gRNA) located immediately upstream of a compatible protospacer adjacent motif (PAM) that may exist on either strand of the DNA helix. Examples of PAM sequence are known (see, e.g., Shah et al., RNA Biology 10 (5): 891-899, 2013).

When the gene editing system is a CRISPR/Cas system, the editing system is used in combination with one or more guide RNAs (gRNAs). For example, the CRISPR/Cas9 system uses an RNA-guide to target Cas9 nuclease to create a double stranded DNA break (DSB) at a specific location. These DSBs are repaired imperfectly, leading to indel formation, which disrupts gene expression. As used herein, a “guide RNA” (gRNA) is nucleotide sequence that is complementary to at least a portion of a target gene. In some embodiments, the sequence of PAM is dependent upon the species of Cas nuclease used in the architecture. It should be noted that the DNA-targeting sequence may or may not be 100% complementary to the target polynucleotide (e.g., gene) sequence. In certain embodiments, the DNA-targeting sequence is complementary to the target polynucleotide sequence over about 8-25 nucleotides (nts), about 12-22 nucleotides, about 14-20 nts, about 16-20 nts, about 18-20 nts, or about 8, 9, 10, 11, 12, 13, 14, 15, 16, 17, 18, 19, 20, 21, 22, 23, 24, or 25 nts. In certain embodiments, the complementary region comprises a continuous stretch of about 12-22 nts, preferably at the 3′ end of the DNA-targeting sequence. In certain embodiments, the 5′ end of the DNA-targeting sequence has up to 8 nucleotide mismatches with the target polynucleotide sequence. In certain embodiments, the DNA-binding sequence is about 50, 55, 60, 65, 70, 75, 80, 85, 90, 95, or 100% complementary to the target polynucleotide sequence.

In some embodiments, gene editing system components Cas9 and a guide RNA (gRNA) comprising a targeting domain, which targets a region of the genetic locus, are introduced into the cell. In some embodiments, the gene editing system components comprise a ribonucleoprotein (RNP) complex of a Cas9 polypeptide and a gRNA (Cas9/gRNA RNP).

It will be understood that CRISPR-Cas systems as described herein are non-naturally occurring in a cell, i.e., engineered or exogenous to the cell, and are introduced into a cell. Methods for introducing the CRISPR-Cas system in a cell are known in the art, and are further described herein elsewhere. The cell comprising the CRISPR-Cas system, or having the CRISPR-Cas system introduced, according to the invention comprises or is capable of expressing the individual components of the CRISPR-Cas system to establish a functional CRISPR complex, capable of modifying (such as cleaving) a target DNA sequence. Accordingly, as referred to herein, the cell comprising the CRISPR-Cas system can be a cell comprising the individual components of the CRISPR-Cas system to establish a functional CRISPR complex, capable of modifying (such as cleaving) a target DNA sequence. Alternatively, as referred to herein, and preferably, the cell comprising the CRISPR-Cas system can be a cell comprising one or more nucleic acid molecule encoding the individual components of the CRISPR-Cas system, which can be expressed in the cell to establish a functional CRISPR complex, capable of modifying (such as cleaving) a target DNA sequence.

For the methods described herein, gene editing systems or components thereof (e.g., a nucleobase editor protein, a gRNA) are introduced into a cell (e.g., a γδ T cell). As used herein, the term “introducing” encompasses a variety of methods of introducing DNA into a cell, either in vitro or in vivo, such methods including transformation, transduction, transfection (e.g. electroporation), nucleofection (an electroporation-based transfection method which enables transfer of nucleic acids such as DNA and RNA into cells by applying a specific voltage and reagents) and infection. Where the introducing involves electroporation (e.g., nucleofection), a polynucleotide (e.g., a plasmid, a single stranded DNA, a minicircle DNA, RNA) is electroporated into a target cell. Vectors are useful for introducing DNA encoding molecules into cells. Any appropriate delivery vector can be used with the methods described herein. For example, delivery vectors include exosomes, viruses (viral vectors), and viral particles. Preferably, the delivery vector is a viral vector, such as a lenti- or baculo- or preferably adeno-viral/adeno-associated viral (AAV) vectors, but other non-viral means of delivery are known (such as yeast systems, microvesicles, gene guns/means of attaching vectors to gold nanoparticles). Other methods of introducing a nucleic acid into a host cell are known in the art, and any known method can be used to introduce a nucleic acid (e.g., vector or expression construct) into a cell for the methods provided herein. Suitable methods include, include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al., Adv. Drug Deliv. Rev.), and the like.

Methods and techniques for assessing the expression and/or levels of cell markers are known in the art. Antibodies and reagents for detection of such markers are well known in the art, and readily available. Assays and methods for detecting such markers include, but are not limited to, flow cytometry, including intracellular flow cytometry, ELISA, ELISPOT, cytometric bead array or other multiplex methods, Western Blot and other immunoaffinity-based methods. In some embodiments, the modified cells can be detected by flow cytometry or other immunoaffinity based method for expression of a marker unique to such cells, and then such cells can be co-stained for another marker.

In some cases, a guide RNA for CRISPR/Cas9-mediated gene editing comprises a modified linkage for stability. For example, a gRNA may be stabilized by one or more phosphorothioate internucleotide linkages and/or 2′-O-methyl modifications at the 3′ and/or 5′ ends. The term “phosphorothioate internucleotide linkage” as used herein refers to internucleotide linkages in which one of the non-bridging oxygens in the DNA phosphate backbone is replaced by sulfur. As used herein, the term “2′-O-methyl modification” refers to nucleotide modifications wherein a methyl group is added to the 2′-hydroxyl group of the ribose moiety of a nucleoside.

Methods of Using Genetically Modified GDTCs

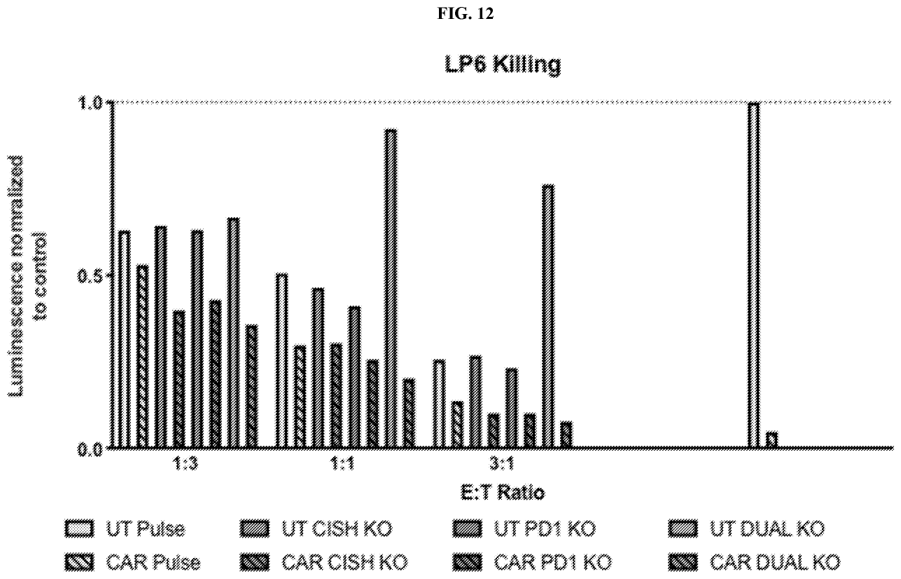

In another aspect, provided herein are methods for using the genetically modified γδ T cells described herein. For example, genetically modified γδ T cells or γδ T cell subsets obtainable by the methods disclosed herein may be used for subsequent steps such as research, diagnostics, pharmacological or clinical applications known to the person skilled in the art. In some cases, genetically modified γδ T cells may be used to treat or prevent a disease or condition in a subject. In some cases, the method comprises introducing a nucleic acid encoding a chimeric antigen receptor (CAR) into a genetically modified γδ T cell, where the CAR has specificity for a surface antigen of a tumor cell and the ability to activate a T cell, expanding a culture of the genome-edited γδ T cells ex vivo, and then administering the genome-edited γδ T cells into a patient. Preferably, the genome-edited γδ T cells are obtained according to the methods described herein. The disease could include, for example, cancer, a precancerous condition, infection with a pathogen (including, for example, malaria), or a viral infection. In some cases, the genetically modified γδ T cells of this disclosure have an increased capacity to treat various cancer types including, without limitation, leukemia, neuroblastoma, and carcinomas, but are modified to reduce the likelihood of uncontrolled inflammation and associated unwanted tissue destruction which may be linked to γδ T-cell-based therapy. For example, FIG. 12 demonstrates cytotoxicity of genetically modified γδ T cells that are CRISPR/Cas9 engineered and express a CAR having specificity for mesothelin. In example of FIG. 12, the engineered γδ T cells were electroporated with Cas9 mRNA and sgRNA targeting either PD1 or CISH alone, or PD1 and CISH combined, and then expanded and co-cultured with mesothelin-expressing cancer cells at the indicated effector to target (E:T) ratios. These data demonstrate that γδ T cells having a genetic modification in PD1 or CISH, or genetic modifications in both PD1 and CISH exhibit increased cytoxicity to cancer cells as determined by loss of reporter expression in the cancer cell line.

In some embodiments, it is preferred that the cells are used for cancer immunotherapy. Advantageously, γδ T cell-mediated cytotoxicity does not rely on the presentation of self-human leukocyte antigens and they are not involved in graft-versus-host disease (GVHD). Accordingly, γδ T cells of this disclosure have a high potential for off-the-shelf immunotherapies. In some cases, for example, γδ T cells can be produced from healthy patients and given to patients whose immune systems are too compromised to be receptive to more conventional immunotherapies. Such allogenic immunotherapies are not limited by donor-matching.

In some cases, γδ T cells genetically modified as described herein can be used to treat various conditions including cancer. For example, γδ T cells obtained as described herein can be used to provide immunotherapy to a subject. Generally, the method comprises administering to a subject in need thereof a therapeutic composition comprising CAR-expressing γδ T cells in which the antigen recognition region of the chimeric antigen receptor specifically binds to an antigen associated with the condition (e.g., particular cancer or tumor type). To “treat” a disease as the term is used herein, means to reduce the frequency or severity of at least one sign or symptom of a disease or disorder experienced by a subject. As used herein, the term “therapeutic” means a treatment and/or prophylaxis. A therapeutic effect is obtained by suppression, remission, or eradication of a disease state.

In some cases, the condition is cancer or a precancerous condition. The cancer may include, for example, bone cancer, brain cancer, breast cancer, cervical cancer, cancer of the larynx, lung cancer, pancreatic cancer, prostate cancer, skin cancer, cancer of the spine, stomach cancer, uterine cancer, hematopoietic cancer, and/or lymphoid cancer, etc. A hematopoietic cancer and/or lymphoid cancer may include, for example, acute myelogenous leukemia (AML), acute lymphoblastic leukemia (ALL), myelodysplastic syndromes (MDS), non-Hodgkin lymphoma (NHL), chronic myelogenous leukemia (CIVIL), Hodgkin's disease, and/or multiple myeloma. The cancer may be a metastatic cancer. The precancerous condition can be a preneoplastic lesion.

In some cases, the γδ T cells are genetically modified ex vivo and contacted to an antigen, polypeptide, or peptide associated with various immunotherapies or gene therapy. In such cases, the modified cells are then returned to the subject as an autologous transplant in advance of the immunotherapy or gene therapy. As used herein, the term “autologous” is meant to refer to any material derived from the same individual to which it is later to be re-introduced into the individual.

In some cases, genetically modified γδ T cells as described herein are provided to a subject in need thereof as a pharmaceutical composition comprising the modified cells and a pharmaceutically acceptable carrier. Carriers which may be used with the genetically modified γδ T cells of the present invention will be well known to those of skill in the art. Methods for formulating the pharmaceutical composition and selecting appropriate doses are well known to those of skill in the art. An appropriate dosage of the pharmaceutical composition of the present invention may be variously prescribed depending on factors such as a formulation method, an administration manner, the age, body weight, sex, administration time and administration route of the patient. The dosage may also depend on the preparation method and yield.

In another aspect, provided herein are methods of targeting a tumor using genetically modified γδ T cells. For example, a genome-edited γδ T cell may be administered to inhibit the growth of a tumor in a subject. In some embodiments, the tumor may include a solid tumor.

The genetically modified γδ T cells and/or γδ T cell subsets can also be used as a pharmaceutical composition in the therapy, e.g. cellular therapy, or prevention of diseases. The pharmaceutical composition may be transplanted into an animal or human, preferentially a human patient. The pharmaceutical composition can be used for the treatment and/or prevention of diseases in mammals, especially humans, possibly including administration of a pharmaceutically effective amount of the pharmaceutical composition to the mammal. Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

The composition of genetically modified γδ T cells obtained by the methods of this disclosure may be administered either alone, or as a pharmaceutical composition in combination with diluents and/or with other components such as cytokines or cell populations. Briefly, pharmaceutical compositions of the present invention may comprise the genome-edited γδ T cells as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

A genome-edited γδ T cell may be administered to a subject before, during, and/or after other treatments. Such combination therapy may involve administering genome-edited γδ T cells before, during and/or after the use of other anti-cancer agents including, for example, a cytokine; a chemokine; a therapeutic antibody including, for example, a high affinity anti-CMV IgG antibody; an antioxidant; a chemotherapeutic agent; and/or radiation. The administration or preparation may be separated in time from the administration of other anti-cancer agents by hours, days, or even weeks. Additionally or alternatively, the administration or preparation may be combined with other biologically active agents or modalities such as, but not limited to, an antineoplastic agent, and non-drug therapies, such as, but not limited to, surgery.

The term “subject” is intended to include living organisms in which an immune response can be elicited or modulated (e.g., mammals). A “subject” or “patient,” as used therein, may be a human or non-human mammal. Non-human mammals include, for example, livestock and pets, such as ovine, bovine, equine, porcine, canine, feline, and murine animals. Accordingly, the term “subject” or “patient” as used herein means any mammalian patient or subject to which the genetically modified cells described herein can be administered. Preferably, the subject is human.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acids and/or other constructs of the invention may be isolated. As used herein, “isolated” means to separate from at least some of the components with which it is usually associated whether it is derived from a naturally occurring source or made synthetically, in whole or in part.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain.

Nucleic acids, proteins, and/or other compositions (e.g., cell population) described herein may be purified. As used herein, “purified” means separate from the majority of other compounds or entities, and encompasses partially purified or substantially purified. Purity may be denoted by a weight by weight measure and may be determined using a variety of analytical techniques such as but not limited to mass spectrometry, HPLC, etc.

In interpreting this disclosure, all terms should be interpreted in the broadest possible manner consistent with the context. It is understood that certain adaptations of the invention described in this disclosure are a matter of routine optimization for those skilled in the art, and can be implemented without departing from the spirit of the invention, or the scope of the appended claims.

So that the compositions and methods provided herein may more readily be understood, certain terms are defined:

As used in this specification and the appended claims, the singular forms “a,” “an,” and “the” include plural references unless the context clearly dictates otherwise. Any reference to “or” herein is intended to encompass “and/or” unless otherwise stated.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The terms “comprising”, “comprises” and “comprised of as used herein are synonymous with “including”, “includes” or “containing”, “contains”, and are inclusive or open-ended and do not exclude additional, non-recited members, elements, or method steps. The phraseology and terminology used herein is for the purpose of description and should not be regarded as limiting. The use of “including,” “comprising,” “having,” “containing,” “involving,” and variations thereof, is meant to encompass the items listed thereafter and additional items. Embodiments referenced as “comprising” certain elements are also contemplated as “consisting essentially of” and “consisting of” those elements. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

Unless otherwise defined, all technical terms used herein have the same meaning as commonly understood by one of ordinary skill in the art to which this invention belongs. As used herein and in the claims, the singular forms “a,” “an,” and “the” include the singular and the plural reference unless the context clearly indicates otherwise. Thus, for example, a reference to “an agent” includes a single agent and a plurality of such agents. Any reference to “or” herein is intended to encompass “and/or” unless otherwise stated.

Various exemplary embodiments of compositions and methods according to this invention in addition to those shown and described herein will become apparent to those skilled in the art from the foregoing description and the following examples and fall within the scope of the appended claims. Those skilled in the art will recognize, or be able to ascertain using no more than routine experimentation, many equivalents to the specific embodiments of the invention described herein. Such equivalents are intended to be encompassed by the following claims.

The present invention has been described in terms of one or more preferred embodiments, and it should be appreciated that many equivalents, alternatives, variations, and modifications, aside from those expressly stated, are possible and within the scope of the invention.

### EXAMPLES

### Example: Efficient Engineering of γδ T Cells Using CRISPR-Cas9 and Functional Effects of Gene Editing in γδ T Cells

Materials and Methods

Stimulation and Expansion of γδ T Cells from PBMCs

In all cases, PBMCs were isolated from peripheral blood using a previously described ACK-lysis buffer protocols. GDTC baseline media (GDTC media) used is in all experiments is OptimizerCTS Medium containing CTS supplement (Gibco). γδ T cells were then obtained and stimulated using 1 of 4 different methods.

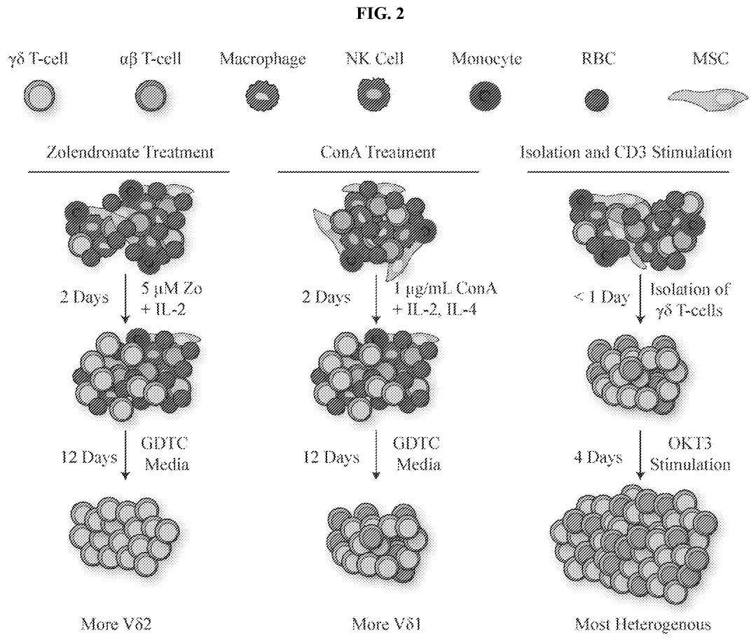

Method 1: γδ T cells were isolated using the EasySep Human Gamma/Delta T Cell Isolation Kit from StemCell Technologies. Isolated cells were treated with 50 μL of CD28/CD3 dynabeads per 1.0×10\hat{ }6 cells in a 24 well plate. Isolated γδ T cells were expanded for 72 hours prior to nucleofection resulting in a 2-4 fold expansion (FIG. 2).

Method 2: γδ T cells were isolated using the EasySep Human Gamma/Delta T Cell Isolation Kit from StemCell Technologies. Isolated cells were then plated into OKT3 coated 24 well plates at a density of 1.0×10″6 cells in a 24 well plate. Isolated γδ T cells were expanded for 72 hours prior to nucleofection resulting in a 2-4 fold expansion (FIG. 3).

Method 3: PBMCs were plated into media containing 1 μg/mL Concanavalin A, 1000 U/mL of IL-2, and 10 ng/mL of IL-4 for 7 days. Cells were then cultured in the same media with no Concanavalin A for an additional 7 days. Percent γδ T cell purity was then determined by flow cytometry.

Method 4: PBMCs were plated into media containing 5 μM Zolendronic Acid and 1000 U/mL of IL-2 for 2 days. Cells were then cultured in the same media with no Zolendronic Acid for an additional 12 days. Percent γδ T cell purity was then determined by flow cytometry.

Gene Knock-Out in γδ T Cells Using CRISPR/Cas9

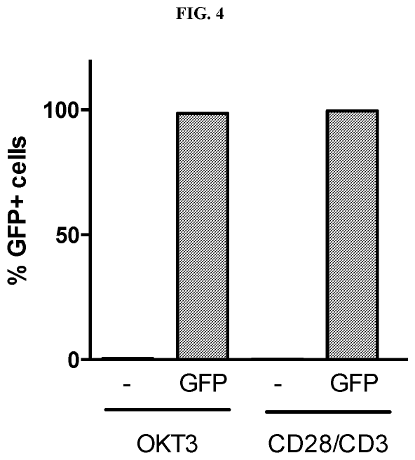

Isolated GDTCs were electroporated using the Lonza Amaxa 4D Nucleofection system with 1 μg of target site guide mRNA and 1.5 μg of Cas9 mRNA 72 hours post-stimulation with either OKT3 or CD28/CD3 dynabeads. Cells were then harvested for genomic DNA isolation and TIDE analysis or flow cytometry 7 days later (FIGS. 4A-4B).

Gene Knock-In in γδ T Cells Using CRISPR/Cas9

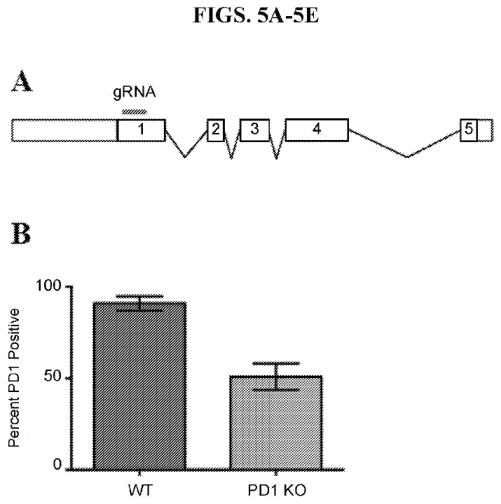

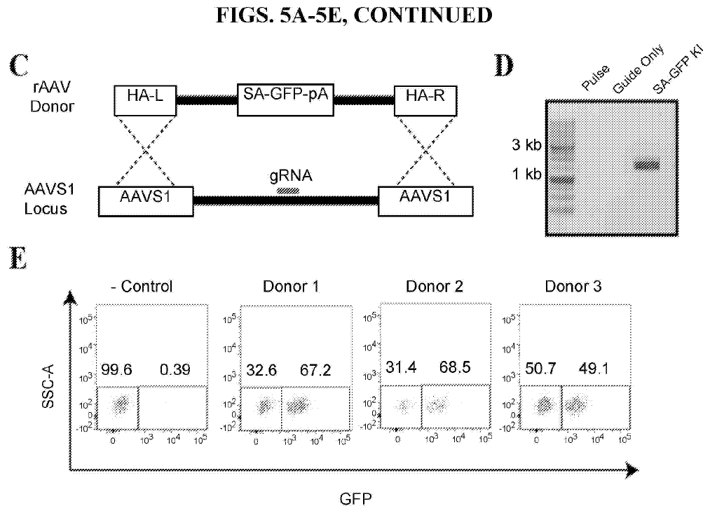

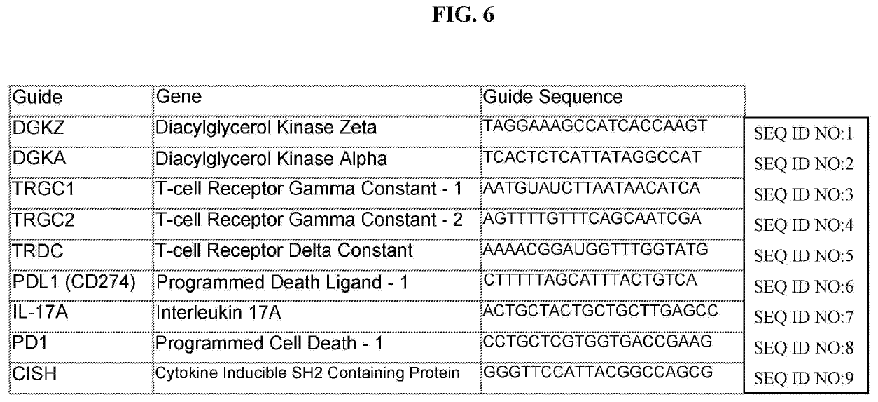

Isolated γδ T cells were electroporated using the Lonza Amaxa 4D Nucleofection system with 1 μg of target site guide mRNA, 1.5 μg of donor DNA, and 1.5 μg of Cas9 mRNA 72 hours post-stimulation with either OKT3 or CD28/CD3 dynabeads. Cells were then harvested for genomic DNA isolation and TIDE analysis or Flow cytometry 7 days later (FIGS. 5C-5E).

Results and Discussion

These data show a novel method for highly efficient engineering of γδ T cells using CRISPR-Cas9. With the protocols described herein, it is possible to consistently achieve a targeting efficiency of about 90% in knockout targeted cell populations, and 65% in knock-in targeted cell populations (FIGS. 5B and 5E). Electroporation efficiency increased by electroporating γδ T cells during their logarithmic growth phase (about 72 hours post stimulation). In fact, 99% of cells electroporated with GFP mRNA are GFP positive by flow cytometry 48 hours after electroporation (FIG. 4).

The stimulation of the cells prior to electroporation additionally results in an increased metabolic activity and therefore, higher levels of Cas9 mRNA translation. This increase in Cas9 protein levels in the cell is likely crucial to achieving high levels of gene targeting as is shown by the increased targeting efficiency in cells electroporated 72 hours after stimulation. In addition to an active metabolic state, a strong stimulus is necessary for the cells to be efficiently targeted.

While a single stimulation of CD3 is sufficient to send the γδ T cell population into cell-cycle, it is not sufficient to increase the metabolic state of the cell enough to achieve the highest efficiency of gene targeting (FIG. 5B). However, when you add an additional stimulation signal such as CD28/CD3 stimulation, the cells are more robustly stimulated, providing for improved gene targeting results (FIG. 5B). Our protocol for gene targeting within γδ T cells dramatically reduces the production time. In particular, with such high levels of gene targeting efficiency, our protocol eliminates the time needed to grow out small targeted populations using traditional gene targeting protocols that require the use of multiple selection markers.

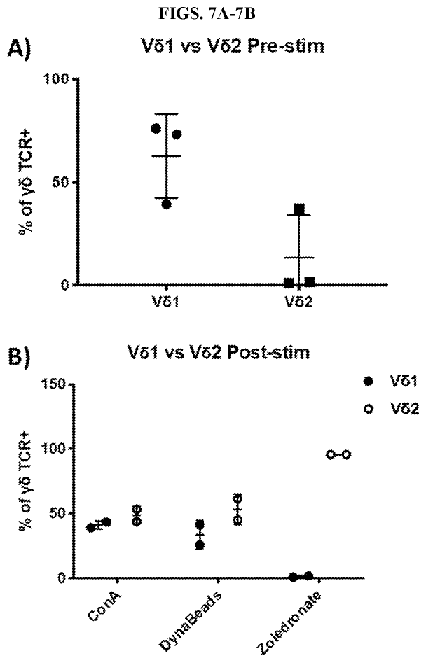

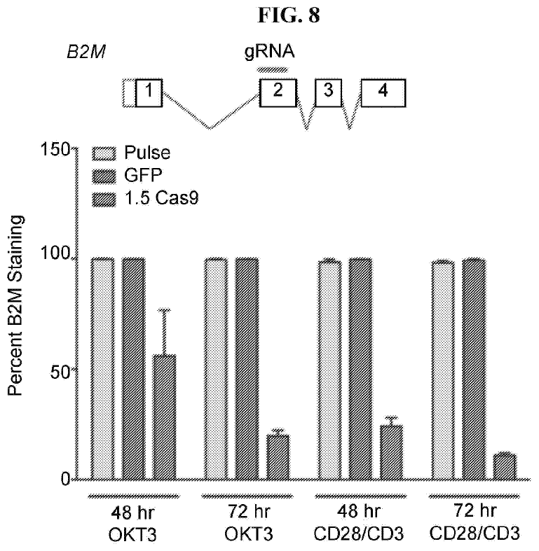

Peripheral blood γδ T cells have varying frequencies of the subpopulations Vd1 and Vd2. We analyzed these subsets by flow cytometry in three independent donors (FIG. 7A). We then stimulated the cells using three different methods concanavalin A (ConA), anti-CD3/CD28 DynaBeads, or Zoledronate to make them amenable to nucleic acid delivery. We analyzed the Vd1 and Vd2 populations after stimulation to see if the stimulation method favored outgrowth of either subset (FIG. 7B). We found that stimulation with ConA or DynaBeads maintained the frequency of the two populations, while Zoledronate stimulation favored the outgrowth of Vd2.

Several γδ T CELL stimulation protocols were tested for optimal nucleic acid delivery and gene editing (FIG. 8). γδ T cells were stimulated for either 48 or 72 hours with anti-CD3 (OKT3) or anti-CD3/CD28 (DynaBeads), before delivering a guide RNA targeting exon 2 of the B2M gene in combination with GFP mRNA (control) or Cas9 mRNA. Gene knockout and protein loss efficiency was assessed by performing flow cytometry staining for the B2M protein. It was determined that stimulation with DynaBeads for 72 hours before gene delivery led to the most efficient gene editing (90% protein loss). Thus, these data demonstrate efficient gene editing in γδ T cells.

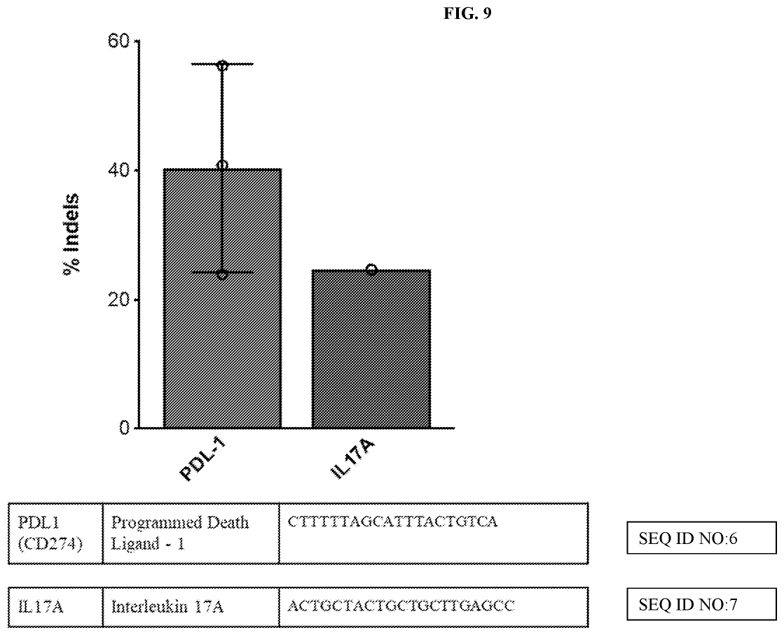

Using the method developed in FIG. 8, guide RNAs targeting immunosuppressive molecules IL17A and PD-L1 were designed and delivered to γδ T cells. Gene editing was analyzed at these two target sites using Sanger sequencing (FIG. 9).

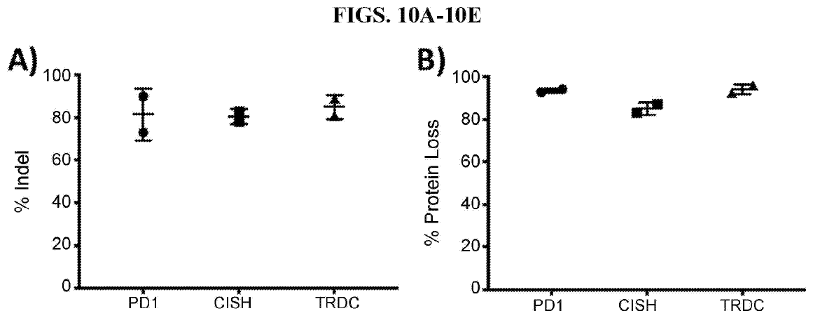

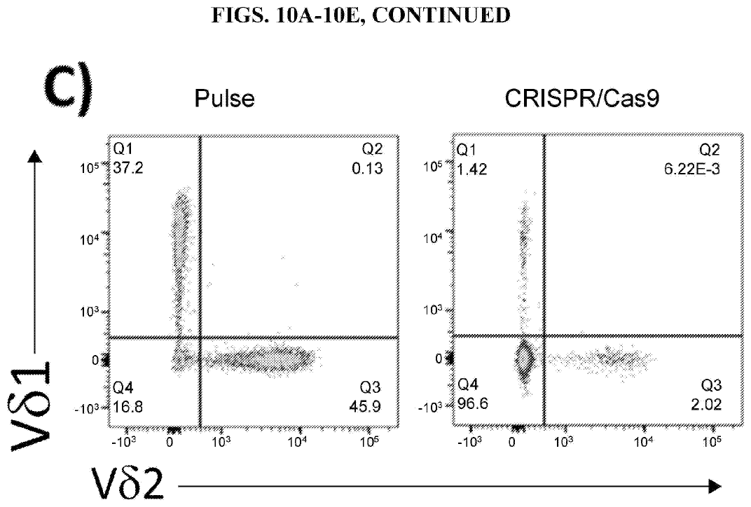

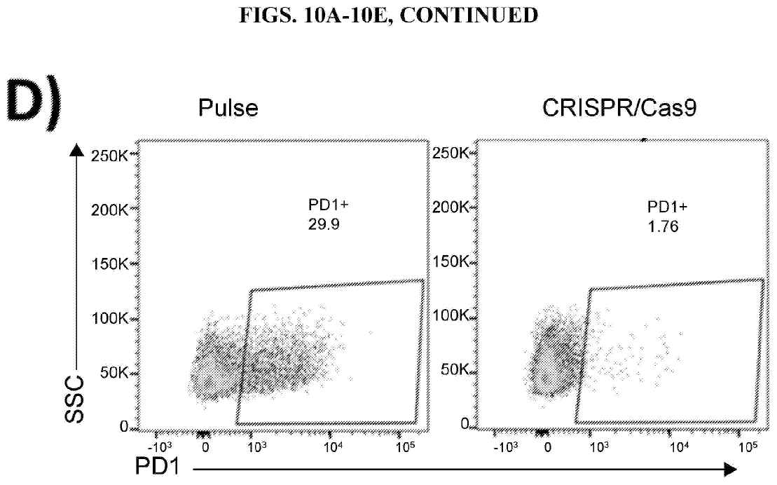

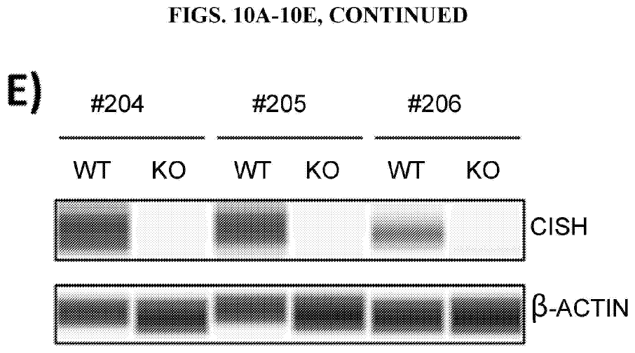

Tumor cells have developed methods for evading detection by immune cells. One such way is by engaging immune checkpoint molecules on immune cells in the tumor microenvironment. PD1 is a negative regulator of T cell function and its cognate receptor, PD-L1, is upregulated in many adult and pediatric cancers. CISH is a negative regulator of immune cell activation and integrates signaling via cytokines, including IL-15. Knockout of these regulatory proteins is predicted to enhance the antitumor efficacy of γδ T cells. TRDC codes for the delta chain of the γδ T CELL receptor. We targeted this gene in preparation for targeted gene delivery to this locus. We designed and delivered guide RNAs targeting these functionally relevant genes in γδ T cells. We confirmed gene editing at both the genomic and protein level (FIGS. 10A-10E). Using our optimized methods we demonstrate highly efficient gene editing and protein loss for all three targets. Thus, we have demonstrated our ability to efficiently target therapeutically relevant genes in γδ T cells.

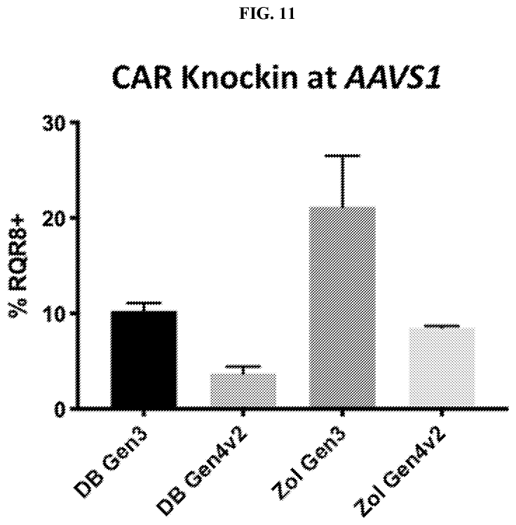

Beyond gene knockout, we have delivered chimeric antigen receptors (CARs) to γδ T cells. CARs will allow γδ T cells to become activated in response to tumor specific antigens. We delivered a CAR targeting the tumor specific antigen Mesothelin, commonly expressed in ovarian and other epithelial cancers. We targeted this construct to the safe harbor locus AAVS1 using a recombinant adeno-associated virus serotype 6 (rAAV6). We delivered two versions of the CAR—one with T-cell specific signaling domains (gen3) and one with NK-cell specific signaling domains (gen4v2) as γδ T cells have characteristics of both T- and NK-cells (FIG. 11). In either DynaBead- or Zoledronate-stimulated γδ T cells, between 5-20% targeted knock-in of the CARs was achieved.

To show a functional effect of gene editing in γδ T cells, a killing assay was performed against the Mesothelin-expressing ovarian cancer cell line A1847. Killing by γδ T cells with or without Mesothelin-CAR expression, with targeted knockout of CISH and/or PD1 (FIG. 12) was compared. Overall, we observed enhanced killing by CAR-expressing γδ T cells.

1. A method for editing a genome of an activated γδ T cells, the method comprising
a) providing a cell sample comprising T cells, T cell subsets and/or T cell progenitors;
b) separating γδ T cells or a γδ T cell subset to thereby provide enriched γδ T cells;
c) activating enriched γδ T cells using one or more modulatory agents to thereby provide activated γδ T cells; and
d) genetically modifying the activated γδ T cells to thereby provide genetically modified T cells comprising one or more modifications in at least one gene selected from IL-17A (Interleukin 17A), DGKA (Diacylglycerol Kinase Alpha), DGKZ (Diacylglycerol Kinase Zeta), PD1 (programmed cell death 1), TRGC1 (T-cell Receptor Gamma Constant-1), TRGC2 (T-cell Receptor Gamma Constant-2), TRDC (T-cell Receptor Delta Constant), PD-L1 (Programmed death-ligand 1), and CISH (Cytokine-inducible SH2-containing protein), or any combination thereof.

2. The method of claim 1, further comprising
e) expanding the genetically modified γδ T cells to thereby provide an expanded population of genetically modified γδ T cells.

3. The method of claim 1, wherein the one or more modulating agents are selected from CD28, CD3, and Concanavalin A.

4. The method of claim 1, wherein the genetically modified γδ T cells further comprise a chimeric antigen receptor comprising an extracellular domain capable of binding to an antigen, a transmembrane domain, and at least one intracellular domain.

5. The method of claim 4, wherein the antigen is a tumor antigen.

6. The method of claim 4, wherein the extracellular domain capable of binding to an antigen is a single chain variable fragment of an antibody that binds to the antigen.

7. The method of claim 1, wherein genetically modifying comprises introducing a nuclease or a nucleic acid encoding a nuclease into the γδ T cell.

8. The method of claim 7, wherein the nuclease comprises Cas9.

9. The method of claim 1, wherein genetically modifying comprises introducing a chemically modified guide RNA (gRNA) into the γδ T cell.

10. The method of claim 9, wherein the chemically modified gRNA comprises 2′-O-methyl (M), 2′-O-methyl-3′-phosphorothioate (MS), or 2′-O-methyl-3′-thiophosphonoacetate (MSP).

11. A genome-edited γδ T cell comprising one or more mutations in a gene selected from IL-17A (Interleukin 17A), DGKA (Diacylglycerol Kinase Alpha), DGKZ (Diacylglycerol Kinase Zeta), PD1 (programmed cell death 1), TRGC1 (T-cell Receptor Gamma Constant-1), TRGC2 (T-cell Receptor Gamma Constant-2), TRDC (T-cell Receptor Delta Constant), PD-L1 (Programmed death-ligand 1), and CISH (Cytokine-inducible SH2-containing protein), or any combination thereof.

12. The genome-edited γδ T cell of claim 11, further comprising a chimeric antigen receptor comprising an extracellular domain capable of binding to an antigen, a transmembrane domain, and at least one intracellular domain.

13. The genome-edited γδ T cell of claim 12, wherein the antigen is a tumor antigen.

14. The genome-edited γδ T cell of claim 12, wherein the extracellular domain capable of binding to an antigen is a single chain variable fragment of an antibody that binds to the antigen.

15. The genome-edited γδ T cell of claim 11, wherein the gene is deleted.

16. The genome-edited γδ T cell of claim 11, wherein the gene comprises a point mutation.

17. The genome-edited γδ T cell of claim 11, further comprising an exogenous gene.

18. The genome-edited γδ T cell of claim 1, wherein the genome-edited γδ T cell exhibits increased capacity to kill cancer cells relative to a non-genome-edited γδ T cell.

19. A method for treating or preventing a disease in a subject, the method comprising: administering to the subject a composition comprising the genome-edited γδ T cell of claim 11.

20. The method of claim 19, wherein the disease comprises cancer or a precancerous condition.


SEQUENCE LISTING (9 sequences)

SEQ ID NO: 1 - DNA, 20 bp, Artificial Sequence
  DGKZ guide sequence
taggaaagccatcaccaagt

SEQ ID NO: 2 - DNA, 20 bp, Artificial Sequence
  DGKA guide sequence
tcactctcattataggccat

SEQ ID NO: 3 - DNA, 20 bp, Artificial Sequence
  TRGC1 guide sequence
aatguaucttaataacatca

SEQ ID NO: 4 - DNA, 20 bp, Artificial Sequence
  TRGC2 guide sequence
agttttgtttcagcaatcga

SEQ ID NO: 5 - DNA, 20 bp, Artificial Sequence
  TRDC guide sequence
aaaacggauggtttggtatg

SEQ ID NO: 6 - DNA, 20 bp, Artificial Sequence
  PDL1 guide sequence
ctttttagcatttactgtca

SEQ ID NO: 7 - DNA, 21 bp, Artificial Sequence
  IL-17A guide sequence
actgctactgctgcttgagcc

SEQ ID NO: 8 - DNA, 20 bp, Artificial Sequence
  PD1 guide sequence
cctgctcgtggtgaccgaag

SEQ ID NO: 9 - DNA, 20 bp, Artificial Sequence
  CISH guide sequence
gggttccattacggccagcg